(19) United States
(12) Patent Application Publication
(10) Pub. No.: US 2009/0191167 A1
(43) Pub. Date: Jul. 30, 2009

(54) ADULT SERTOLI CELLS AND USES THEREOF

(75) Inventor: David J. White, London (CA)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Sertocell Biotechnology (US) Corp., Reno, NV (US)

(21) Appl. No.: 12/375,159

(22) PCT Filed: Jul. 27, 2007
(86) PCT No.: PCT/US07/74623

§ 371 Date: Feb. 27, 2009

Related U.S. Application Data
(60) Provisional application No. 60/820,760, filed on Jul. 28, 2006.

(51) Int. Cl. A61K 35/12; A61P 3/10

(52) U.S. Cl. 424/93.7

(57) ABSTRACT

The invention relates, in part, to non-neonatal Sertoli cells derived from non-rodent animals, pharmaceutical compositions comprising such Sertoli cells, and uses thereof. The non-neonatal, non-rodent Sertoli cells express more FasL than neonatal Sertoli cells, and they provide greater immunoprivilege than neonatal Sertoli cells. In some embodiments the Sertoli cells are modified to express a biological factor. In other embodiments, the pharmaceutical compositions further comprise non-Sertoli cells. The invention also provides implantation devices comprising the pharmaceutical compositions, methods of making the pharmaceutical compositions, and methods of using the pharmaceutical compositions by administering an effective amount of the compositions.

This application claims priority from, and the benefit of, U.S. provisional patent application No. 60/820,760, filed on Jul. 28, 2006, which is incorporated herein by reference.

### TECHNICAL FIELD

The invention relates to the field of tissue and cell transplantation as well as therapeutic methods that involve administration of cells to a subject. More particularly, the invention relates to Sertoli cells.

### BACKGROUND OF THE INVENTION

Some areas of the body, such as the eye, brain, and testis, can limit or prevent the immune response—a phenomenon known as immune privilege. Sertoli cells comprise a major component of the mammalian testis and are responsible for providing immune privilege. Sertoli cells are considered to be “nurse” or “chaperone” cells because they immunoprotect and assist in the development of germ cells into spermatozoa. The immune privilege function is critical for the germ cells because they express cell surface markers that are otherwise recognized as foreign by the subject's immune system. The immune system becomes competent during the peri-natal phase of development and at that time “learns” to recognize all present antigens as “self”. Since germ cells develop after puberty, they express new antigens that are not recognized as “self” by the immune system. Thus, without the ability of Sertoli cells to protect germ cells from the immune system, the germ cells would be destroyed.

For a review on Sertoli cells, see, e.g., Sertoli Cell Biology, Skinner and Griswold (eds.), Elsevier Academic Press, 2005. Although the immunoprotective properties of Sertoli cells have been studied extensively, the exact mechanism of the immune privilege remains elusive. Evidence suggests that local immune tolerance is at least partially mediated by factors produced and/or secreted by Sertoli cells (Bellgrau et al., Nature (1995) 377:630-2; De Cesaris et al., Biochem. Biohys. Res. Commun. (1992) 186:1639-46; Korbutt et al., Diabetologia (2000) 43:474-80; Selawry et al., Transplant. (1991) 52:846-50; Suarez-Pinzon et al., Diabetes (2000) 49:1810-18; Wyatt et al., J. Reprod. Immunol. (1988) 14:27-40). For example, Sertoli cells are known to produce:

1) CD95 ligand (CD95L, also known as Fas ligand (FasL)), which is thought to have immunoprotective properties (Bellgrau et al., Nature (1995) 377:630-2; Griffith et al., Science (1999) 270:1189-9216; Green et al., Nat. Rev. Mol. Cell. Biol. (2001) 2(12):917-24);

2) transforming growth factor-β (TGF-β), which is thought to have anti-inflammatory properties (Avallet et al., Endocrin. (1994) 134:2079-87; Cupp et al., Biol. Reprod. (1999) 151:17-23; Merly et al., Transplant. (1998) 65:893-799; Wahl et al., Immunol. Today (1989) 10:258-261); and

3) clusterin which is thought to have tolerizing properties (Bailey et al., Mol. Cell. Endocrinol. (1999) 151:17-23; Clark et al., J. Androl. (1997) 18:257-67, Lymar et al., Biol. Reprod. (2000) 63:1341-51; Jenne et al., Proc. Natl. Acad. Sci. USA (1989) 86:7123-27).

In 1993, Selawry et al. reported that Sertoli and islet cells co-transplanted under the kidney capsule of diabetic rats were able to survive indefinitely (Selawry et al., Cell Transplant. (1993) 2:123-9). Since then, significant efforts have been devoted to developing cell therapies involving Sertoli cells, for example, co-grafting of Sertoli cells together with islets for treatment of diabetes, or together with dopaminergic tissues for treatment of Parkinson's disease. A significant amount of evidence has been accumulated indicating that Sertoli cells can engraft and self-protect when transplanted into allogeneic and xenogeneic environments (Beligrau et al., Nature (1995) 377:630-2; Dufour et al., Xenotransplant. (2003) 10:577-586; Gores et al., Transplant. (2003) 75:913-18; Saporta et al., Exp. Neurol. (1997) 146:299-304; Yang et al., Transplant. (1999) 67:815-820; Korbutt et al., Diabetologia (2000) 43:474-80), as well as protect co-transplanted allogeneic and xenogeneic cells from immune-mediated destruction (Dufour et al., Transplant. (2003) 75:1594-6; Korbutt et al., Diabetes (1997) 46:317-22; Sanberg et al., Nat. Biotech. (1996) 14:1692-1695; Selawry et al., Cell Transplant. (1993) 2:123-9; Yang et al., Transplant. (1999) 67:815-20; Isaac et al., Transplant. Proc. (2005) 37(1):487-8; Wang et al., Transplant. Proc. (2005) 37(1):470-1).

Previous studies in rodent models employed Sertoli cells obtained from sexually mature rodents. However, Sertoli cells used in larger animal models were obtained from testes of neonatal animals, for example, pigs, presumably, because of the abundant availability of the source (see, e.g., Isaac et al., Transplant. Proc. (2005) 37(1):487-8; Wang et al., Transplant. Proc. (2005) 37(1):470-1; Dufour et al., Biol. Reprod. (2005) 7(5):1224-31; Valdes-Gonzalez et al., Eur. J. Endocrinol. (2005) 153(3):419-27; Dufour et al., Xenotransplant. (2003) 10(6):577-86).

There continues to be a need to develop new and improved methods of cell therapy, in general, and methods utilizing Sertoli cells, in particular.

### SUMMARY OF THE INVENTION

The invention relates to Sertoli cells and uses thereof. In one embodiment, the invention provides a pharmaceutical composition comprising non-neonatal Sertoli cells derived from a non-rodent animal. The non-neonatal Sertoli cells express more Fas ligand than neonatal Sertoli cells, and they provide greater immunoprivilege than neonatal Sertoli cells.

The invention provides methods of selecting non-neonatal Sertoli cells with increased immunoprotective properties. In some embodiments, the non-neonatal Sertoli cells are adult Sertoli cells. In one embodiment, the non-neonatal, non-rodent Sertoli cells comprise porcine cells, which can be obtained, for example, from adult pigs. In other embodiments, the non-neonatal, non-rodent Sertoli cells can be modified to express a biological factor, such as, e.g., insulin.

In further embodiments, the pharmaceutical composition can additionally comprise non-Sertoli cells. In one embodiment, the non-Sertoli cells are insulin-secreting cells, such as beta cells. In another embodiment, the insulin-secreting cells are cells that have been modified to produce insulin, such as modified hepatocytes or other non-insulin-dependent glucose-responsive cells.

The invention also provides methods of making and using the pharmaceutical compositions, including methods of administering an effective amount of the composition to a subject in need thereof. In some embodiments, the invention provides a method of treating diabetes. The invention also provides an implantation device for administering the pharmaceutical compositions and methods of using the implantation device.

Additional details of the invention are disclosed herein.

### BRIEF DESCRIPTION OF THE DRAWINGS

FIG. 1A is a graph depicting relative CD95L (pFasL) mRNA expression levels in neonatal testicles versus adult testicles, as determined by RT-PCR. Results represent pooled values for 4 different neonatal testicles and 5 separate boar testicles, and cDNA preparations were repeated at least twice for each testicle. All expression levels (cycle thresholds (C_{T})) were first normalized against beta actin expression levels, and expression levels in neonatal testicles were set at 100%. In this example, CD95L expression levels in adult testicles is at least eight-fold higher than in neonatal testicles.

FIG. 1B is a graph depicting relative expression levels of CD95L (FasL) mRNA in Sertoli cells cultured over varying lengths of time as determined by RT-PCR. All expression levels (cycle thresholds (C_{T})) were first normalized against beta actin expression levels, and cells harvested after 2 days of culture were arbitrarily set to 100% for comparison with cells cultured over different time periods. Expression levels in cultures grown in 10% bovine serum in high glucose DMEM media (Boar Day 2 to Boar Day 21) increased over the culture period. The Boar Day 9 (1077) culture was separately prepared using a small cell isolation procedure and demonstrated an increase in CD95L expression compared to the cultures grown in high glucose DMEM media. The Boar Day 25 culture was grown in 15% FetalClone II in DMEM high glucose and demonstrated a loss in CD95L expression.

FIG. 2A is a graph depicting rat anti-pig IgG levels in rat serum as determined using flow cytometry. Serum was collected from rats at various time points before or after transplantation with either 200,000 neonatal Sertoli cells plus 2,000 islet cells, or 200,000 adult Sertoli cells plus 2,000 islet cells. Fluorescence levels in each sample were measured using a Cytomics FC500 flow cytometer (Beckman Coulter). Maximum fluorescence was calculated and results presented as a ratio of this maximum value to the maximum fluorescence of a control serum that was run in each experiment. In this example, anti-pig IgG antibody levels, as indicated by the higher fluorescence ratios, were higher in serum from rats transplanted with islet cells and neonatal Sertoli cells than in serum from rats transplanted with islet cells and adult Sertoli cells.

FIG. 2B is a graph depicting rat anti-pig IgG levels in rat serum as determined using flow cytometry. Serum was collected from rats at various time points before or after transplantation with either 11×10⁶ neonatal Sertoli cells and 2,000 islet cells, or 11×10⁶ adult Sertoli cells and 2,000 islet cells. Fluorescence was measured as described above. In this example, anti-pig IgG antibody levels, as indicated by the higher fluorescence ratios, were higher in serum from rats transplanted with islet cells and neonatal Sertoli cells than in serum from rats transplanted with islet cells and adult Sertoli cells.

FIG. 3A is a micrograph of a section from a chamber used to transplant 2,000 neonatal porcine islets into non-immunosuppressed rats. The chamber was removed 7 days post-transplantation, sectioned, and stained with hematoxylin and eosin. A cellular infiltrate is present in this section.

FIG. 3B is a micrograph of a section from a transplant chamber used to transplant 2,000 neonatal porcine islets plus 200,000 neonatal porcine Sertoli cells into non-immunosuppressed rats. The chamber was removed 7 days post-transplantation, sectioned, and stained with hematoxylin and eosin. A cellular infiltrate is present in this section.

FIG. 3C is a micrograph of a section from a transplant chamber used to transplant 2,000 neonatal porcine islets plus 200,000 adult porcine Sertoli cells into non-immunosuppressed rats. The chamber was removed 7 days post-transplantation, sectioned, and stained with hematoxylin and eosin. No cellular infiltrate is present in this section.

FIG. 4A is a micrograph of a section from a transplant chamber used to transplant 2,000 islets plus 200,000 neonatal Sertoli cells into non-immunosuppressed rats. The chamber was removed 1 week post-transplantation, sectioned, and immunostained for the presence of insulin producing cells. Insulin producing cells are indicated with arrows.

FIG. 4B is a micrograph of a section from a transplant chamber used to transplant 2,000 islets plus 200,000 neonatal Sertoli cells into non-immunosuppressed rats. The chamber was removed 5 weeks post-transplantation, sectioned, and immunostained for the presence of insulin producing cells. Insulin producing cells are indicated with arrows.

FIG. 5 is a micrograph of a section from a transplant chamber used to transplant 2,000 islets plus 200,000 adult Sertoli cells into non-immunosuppressed rats. The chamber was removed 6 weeks post-transplantation, sectioned, and immunostained for the presence of insulin producing cells. Insulin producing cells are indicated with arrows.

FIGS. 6A to 6F are micrographs of sections from transplant chambers used to transplant various combinations of porcine islet cells and porcine Sertoli cells into rats. The chambers were removed 7 days post-transplantation and stained with hematoxylin and eosin. FIGS. 6A to 6C are magnified 50×. FIGS. 6D to 6F are magnified 400×. In FIGS. 6A and 6D, 4,000 islet cells were transplanted in the chambers. In FIGS. 6B and 6E, 4,000 islet cells plus 400,000 neonatal Sertoli cells were transplanted in the chamber. In FIGS. 6C and 6F, 4,000 islet cells plus 400,000 adult Sertoli cells were transplanted in the chamber. Arrows indicate mononuclear cells, which are present in greatest density in FIG. 6D, in lesser density in FIG. 6E, and in least density in FIG. 6F.

FIG. 7 is a micrograph of a section of a chamber used to transplant 4,000 islet cells plus 400,000 adult Sertoli cells into a rat. The section was stained for insulin producing cells by immunohistochemistry. Insulin producing cells appear as dark-stained cells indicated by arrows.

FIGS. 8A to 8F are micrographs of sections from transplant chambers used to transplant various combinations of porcine islet cells and porcine Sertoli cells into rats. The chambers were removed 4 days post-transplantation and stained with hematoxylin and eosin (FIGS. 8A to 8C) or stained for insulin producing cells by immunohistochemistry (FIGS. 8D to 8F). FIGS. 8A to 8C are magnified 50×. FIGS. 8D to 8F are magnified 400×. In FIGS. 8A and 8D, 4,000 islet cells were transplanted in the chambers. In FIGS. 8B and 8E, 4,000 islet cells plus 400,000 neonatal Sertoli cells were transplanted in the chamber. In FIGS. 8C and 8F, 4,000 islet cells plus 400,000 adult Sertoli cells were transplanted in the chamber. Arrows indicate insulin producing cells, which are present in each of FIGS. 8D, 8E, and 8F.

FIGS. 9A to 9F are micrographs of sections from transplant chambers used to transplant various combinations of porcine islet cells and porcine Sertoli cells into rats. The chambers were removed 1 day post-transplantation and stained for insulin producing cells by immunohistochemistry. FIGS. 9A to 9C are magnified 50×. FIGS. 9D to 9F are magnified 200×. In FIGS. 9A and 9D, 4,000 islet cells were transplanted in the chambers. In FIGS. 9B and 9E, 4,000 islet cells plus 400,000 neonatal Sertoli cells were transplanted in the chamber. In FIGS. 9C and 9F, 4,000 islet cells plus 400,000 adult Sertoli cells were transplanted in the chamber. Arrows indicate insulin producing cells, which are present in each of FIGS. 9D, 9E, and 9F.

FIG. 10 is a graph depicting levels of porcine insulin serum from rats transplanted with two transplant chambers, each containing 2,000 neonatal porcine islet cells and 11×10⁶ adult porcine Sertoli cells. Porcine insulin was detected using an ELISA assay. Serum samples were obtained before transplant, and 1 week and 2 weeks post-transplantation. Porcine insulin levels increased post-transplantation.

FIG. 11A depicts Western blots of lysates from neonatal and adult porcine Sertoli cells isolated from testicles. Sertoli cells were isolated from neonatal and adult pig testicles, cultured, and lysed. The lysate proteins were separated by 12% SDS-PAGE, then transferred to PVDV membranes and probed with anti-CD95L (FasL) antibody or β-tubulin antibody. The Western Blots were developed by chemiluminescence. Size markers are indicated on the side of the Western Blots, as are the soluble and membrane-bound forms of CD95L. The membrane-bound form of CD95L in adult tissue has a different mobility compared to that found in neonatal tissue.

FIG. 11B depicts PCR amplified CD95L (FasL) cDNA from neonatal and adult testicular tissue. RNA was isolated and reverse transcribed into cDNA. The cDNA was amplified using primers to either GAPDH or CD95. PCR products were resolved on 12% acrylamide gels and stained with ethidium bromide. Digitized images of the gels were used to quantify band intensities using ImageQuant software. CD95L expression levels were normalized to GAPDH expression levels and compared between neonatal and adult Sertoli tissues. In this case, CD95L is expressed at least 6 fold greater in adult tissue compared to neonatal tissue.

### DETAILED DESCRIPTION OF THE INVENTION

The present invention is based, in part, on the unexpected discovery that Sertoli cells obtained from sexually mature pigs are substantially more immunoprotective compared to Sertoli cells from neonatal pigs. The invention is further based, in part, on the unexpected discovery that the adult Sertoli cells express significantly higher levels of FasL as compared to the neonatal cells. Thus, the use of adult Sertoli cells provides advantages over the use of non-adult Sertoli cells, such as neonatal cells.

Accordingly, the invention provides pharmaceutical compositions comprising non-neonatal, non-rodent Sertoli cells. The Sertoli cells of the invention may provide greater immunoprivilege than neonatal Sertoli cells of the same species.

In some embodiments, the non-neonatal Sertoli cells are such that they express more FasL at the RNA and/or at the protein level(s) than neonatal Sertoli cells of the same species. The cells of the invention may express at least 50% more (e.g., 2, 3, 4, 5, 6, 7, 8, 9, 10 times more or greater) FasL at the RNA and/or at the protein level(s) as compared to neonatal Sertoli cells from the same species.

In some embodiments, the invention provides a method of making a pharmaceutical composition, which comprises isolating Sertoli cells from non-neonatal, non-rodent mammals. For example, the cells may be isolated from a non-neonatal pig that is at least 1, 2, 3, 6, 7, 8, 9, 12, 18, or 24 months old or older.

In some embodiments, the non-neonatal Sertoli cells are adult Sertoli cells. The term “adult”, as used herein, refers to the age of a sexually mature male specimen from which the cells are derived. Sexual maturity is the stage at which an organism can reproduce. For example, male pigs reach sexual maturity at 6-9 months of age, male rats reach sexual maturity at 3 months, and male mice reach sexual maturity at 5-7 weeks. In illustrative embodiments, the Sertoli cells are porcine cells derived from about 1 to 2 year old boars. Alternatively, the Sertoli cells of the invention may be obtained from any suitable source, for example, cows, horses, dogs, cats, rabbits, primates (human or non-human (e.g., monkeys, chimpanzees)), etc.

In some embodiments, the Sertoli cells of the invention have one or both characteristics, i.e., a) they are adult cells and/or b) they express elevated levels of FasL.

The isolated Sertoli cells may and often do contain other cell types naturally present in the testes, including endothelial cells, Leydig cells, etc. Accordingly, pharmaceutical compositions of the invention may further comprise non-Sertoli cells, including cells that are naturally present in the testes and are, therefore, co-isolated with Sertoli cells.

The Sertoli cells of the invention may be primary cells or cell lines derived from such primary cells.

The Sertoli cells of the invention may be genetically altered, for example, they may be genetically modified to express, and optionally, secrete a virus or a biological factor. Examples of such biological factors include insulin, thyroid hormone, neutrophins, Factors VIII and IX, etc. Methods for cell transfection and transformation are known in the art. Methods of gene therapy with Sertoli cells are described, for example, in Dufour et al., Cell Transplant. (2004) 13(1):1-6 and Trivedi et al., Exp. Neurol. (2006) 198, 88-100.

Additionally, pharmaceutical compositions of the invention may comprise non-testicular cells. For example, Sertoli cells may be co-cultured and/or transplanted with another cell type, which benefits from the immunoprotective effect of the Sertoli cells. Specific examples of such other cell types include those that either naturally produce or were modified to produce a desired virus or biological factor, such as those listed above.

The pharmaceutical composition of the invention may further comprise buffers, excipients, inhibitors and preservants, etc.

The invention further provides an implantation device comprising the pharmaceutical composition of the invention. For example, the device may be adapted to induce formation of a fibrotic capsule when implanted into a mammal, as described, e.g., in U.S. Pat. No. 6,716,246. For instance, the device may comprise a mesh chamber containing a removable core (e.g., mechanically removable or biodegradable). The device may be also configured to contain and prevent release of cells into the subject's system but allow for exchange of soluble factors (e.g., to reduce safety risks when using transformed cell lines in therapy).

The invention further provides methods of using the pharmaceutical compositions and devices of the invention. Such methods include administering an effective amount of the composition to a subject (e.g., a non-rodent subject, e.g., human). The effective amount may be, for example, such that it results in the improvement, or slowing in the progression of at least some aspects of disease or an undesirable condition.

The cells may be administered to a subject (e.g., in a device, or as a cell suspension without a device) at a site with or without a pre-implanted device. The cells may be administered, for example, under the kidney capsule, under the skin, or directly into the affected organ or tissue. The Sertoli cells may be autogeneic, allogeneic or xenogeneic to the subject.

In some embodiments, the subject has one or more conditions such as type 2 diabetes, autoimmune disease (e.g., rheumatoid arthritis, lupus, type 1 diabetes), neurodegenerative and neural disorder and conditions (e.g., Parkinson's disease, spinal cord injury), hemophilia, or cancer. Additionally, the methods of the inventions may be used in conjunction with organ or tissue transplantation.

In particular embodiments, the invention provides a method of treating diabetes, comprising co-administering the Sertoli cells of the invention and non-Sertoli insulin-secreting cells (e.g., beta cells in islets) to a mammal in need thereof and under conditions that allow the islet cells to survive and produce insulin subsequent to administration. Alternatively, the Sertoli cells can be co-transplanted with cells that normally do not produce insulin but have been modified to produce it (for example, modified hepatocytes or other non-insulin-dependent glucose-responsive cells, such as, e.g., certain intestinal and kidney cells, and alpha cells).

The invention further provides a method of selecting non-neonatal Sertoli cells with increased immunoprotective properties. The method comprises determining the amount of FasL expressed by the Sertoli cells, and selecting cells expressing higher amounts of FasL. The method for determining the expression levels of FasL are known in the art and include, e.g., FACS, RT-PCR. Illustrative methods are described in the Examples below.

Methods of isolating and other methods of using Sertoli cells are known in the art and illustrative methods are described in the Examples below. Additional methods of making and methods of using Sertoli cells, including various therapeutic indications and devices for use with the cells, are described in the following patent documents: WO 95/28167, WO 96/28174, WO 98/28030, WO 00/27409, WO 2000/035371, WO 2005/018540, U.S. Pat. No. 5,725,854, U.S. Pat. No. 5,843,340, U.S. Pat. No. 5,849,285, U.S. Pat. No. 5,948,422, U.S. Pat. No. 5,958,404, U.S. Pat. No. 6,149,907, U.S. Pat. No. 6,303,355, U.S. Pat. No. 6,649,160, U.S. Pat. No. 6,716,246, U.S. Pat. No. 6,783,964, U.S. Pat. No. 6,790,441, U.S. Pat. No. 6,958,158, US Pat. App. Pub. 2005/0118145.

The following references provides additional details:
1. Valdes-Gonzalez, R. A. et al. Xenotransplantation of porcine neonatal islets of Langerhans and Sertoli cells: a 4-year study. Eur J Endocrinol 153, 419-427 (2005).
2. Basta, G. et al. Transdifferentiation molecular pathways of neonatal pig pancreatic duct cells into endocrine cell phenotypes. Transplant Proc 36, 2857-2863 (2004).
3. Balkan, W., Oates, E. L., Howard, G. A. & Roos, B. A. Testes exhibit elevated expression of calcitonin gene-related peptide receptor component protein. Endocrinology 140, 1459-1469 (1999).
4. Nehar, D., Mauduit, C., Boussouar, F. & Benahmed, M. Tumor necrosis factor-alpha-stimulated lactate production is linked to lactate dehydrogenase A expression and activity increase in porcine cultured Sertoli cells. Endocrinology 138, 1964-1971 (1997).
5. Carstensen, J. F. et al. Characterization of 17 beta-hydroxysteroid dehydrogenase IV. J Endocrinol 150 Suppl, S3-12 (1996).
6. Selawry, H. P., Wang, X. & Alloush, L. Sertoli cell-induced defects on functional and structural characteristics of isolated neonatal porcine islets. Cell Transplant 5, 517-524 (1996).
7. Avallet, O., Vigier, M., Leduque, P., Dubois, P. M. & Saez, J. M. Expression and regulation of transforming growth factor-beta 1 messenger ribonucleic acid and protein in cultured porcine Leydig and Sertoli cells. Endocrinology 134, 2079-2087 (1994).
8. Risbridger, G. P., Robertson, D. M. & de Kretser, D. M. Current perspectives of inhibin biology. Acta Endocrinol (Copenh) 122, 673-682 (1990).
9. Sano, A., Radin, N. S., Johnson, L. L. & Tarr, G. E. The activator protein for glucosylceramide beta-glucosidase from guinea pig liver. Improved isolation method and complete amino acid sequence. J Biol Chem 263, 19597-19601 (1988).
10. Schneyer, A. L., Reichert, L. E., Jr., Franke, M., Ryan, R. J. & Sluss, P. M. Follicle-stimulating hormone (FSH) immunoactivity in porcine follicular fluid is not pituitary FSH. Endocrinology 123, 487-491 (1988).
11. Chatelain, P. G., Naville, D. & Saez, J. M. Somatomedin-C/insulin-like growth factor 1-like material secreted by porcine Sertoli cells in vitro: characterization and regulation. Biochem Biophys Res Commun 146, 1009-1017 (1987).
12. Benahmed, M., Morera, A. M. & Chauvin, M. A. Evidence for a Sertoli cell, FSH-suppressible inhibiting factor(s) of testicular steroidogenic activity. Biochem Biophys Res Commun 139, 169-178 (1986).
13. Renier, G. et al. Isolation, purification and culture of Sertoli cells from immature piglet testes. Acta Endocrinol (Copenh) 111, 411-418 (1986).
14. Perrard, M. H., Saez, J. M. & Dazord, A. Effects of FSH on acidic nuclear protein synthesis in cultured pig Sertoli cells. FEBS Lett 168, 49-53 (1984).
15. Feig, L. A., Klagsbrun, M. & Belive, A. R. Mitogenic polypeptide of the mammalian seminiferous epithelium: biochemical characterization and partial purification. J Cell Biol 97, 1435-1443 (1983).
16. Valdes-Gonzalez, R., Silva-Torres, L., Ramirez-Gonzalez, B., Ormsby, C. E., Teran-Ortiz, L., Ayala-Sumuano, J. T., Method for evaluating quality of cultured neonatal pig Sertoli cells. Xenotransplant 12(4):316-23 (2005).
17. Dufour J M, Gores P, Hemendinger, R., Emerich, D. F., Halberstadt, C. R., Transgenic Sertoli cells as a vehicle for gene therapy. Cell Transplant 13(1):1-6 (2004).
18. Sanberg, P. R., et al. Testis-derived Sertoli cells have a trophic effect on dopamine neurons and alleviate hemiparkinsonism in rats. Nat Med 3, 1129-32 (1997).
19. Willing, A. E., et al. Sertoli cells enhance the survival of co-transplanted dopamine neurons. Brain Res 822, 246-50 (1999).

The following Examples are intended for illustrative purposes and do not limit the invention as claimed.

### EXAMPLES

### Example 1

### Sertoli Cells from Adult Pigs Express Substantially More CD95L (FasL) than Those from Neonatal Pigs

Sertoli Cell Culture—Isolated Sertoli cells were seeded at 5×10⁵ in 25 cm² collagen culture flasks (Falcon) with 10% FetalClone II in DMEM high glucose (Hyclone) or 10% bovine serum (Sigma) in DMEM high glucose with 1% penicillin/streptomycin (Sigma). Cell cultures were maintained in low oxygen (5%) in a 37° C. humidified incubator with 5% CO₂. Samples were taken at each passage for three weeks and examined for CD95L expression levels.

Small cell Isolation—isolated Sertoli cells (4×10⁸) were cultured in 175 cm² non-collagen flasks (Falcon), with 10% bovine serum in DMEM high glucose. After two days, chains of small (˜2-5 μm) cells appeared and these were separated using standard gradient centrifugation (Histopaque 1077, Sigma). Isolated cells were then seeded back onto collagen coated flasks and examined as above for CD95L expression levels.

Testicle RNA Isolation—Fresh testes from 18-21 day old piglets and boars of different breeds (including Duroc and Large White) ranging from 1 to 2 years in age were cut into two pieces and a central thin section removed and weighed. Homogenization was performed by freeze-thaw and passage through a fine 23 gauge needle and then through a QlAshredder (Qiagen). RNA was isolated using the RNeasy mini kit as per manufacturer's instructions (Qiagen). RNA was quantified on a Beckman Coulter DU530 spectrophotometer at 260 nm.

RealTime PCR Expression profiles—RealTime PCR was carried out in triplicate with 100 ng of reverse transcribed total RNA in an MX4000 (Stratagene). Briefly, 1.5 μg of total RNA was transcribed using Stratascript reverse transcriptase in a 30 μL volume utilizing random hexamers as directed by the manufacturer (Stratagene). For RealTime PCR, a SYBR green master mix kit (Stratagene) was employed in all reactions according to the manufacturer in a 30 μL reaction volume and included a ROX reference dye (Stratagene). Standardized cycling parameters were as follows: 10 min at 95° C. followed by 40 cycles at 95° C. for 30 s, 60° C. for 30 s and 72° C. for 1 min. Data was collected for analysis at 72° C. Primers were added to a final concentration of 250 nM and included the following sets designed across exon-intron boundaries from published sequences. Porcine FasL was quantified using three different primer sets: qF1: 5′ACT GAA CTC AGA GAG TCT GCC AGC C (SEQ ID NO:1) and qR1: 5′GGA TGG ATC TTG AGT TAG GCT TGC C (SEQ ID NO:2); qF2: 5′TGA TGT TCT TCA TGG TTC TGG TGG C (SEQ ID NO:3) and qR2: 5′GCT TCT CCA AAG ATG ATT CTG TAT GCC T (SEQ ID NO:4); qF3: 5′TCT TCC ACC TAC AGA AGG AGC TGA CTG (SEQ ID NO:5) and qR3: CCA TTC CAG AGG GAT GGA TCT TGA G (SEQ ID NO:6). Products were analyzed by melting curve determinations. All primers generated single products and closely matched the predicted melting temperatures. Beta actin expression levels were utilized to normalize expression between experiments and animals using the primer set: TTG CCG ACA GGA TGC AGA AGG (forward) (SEQ ID NO:7) and GAC AGC GAG GCC AGG ATG GAG (reverse) (SEQ ID NO:8).

### Results

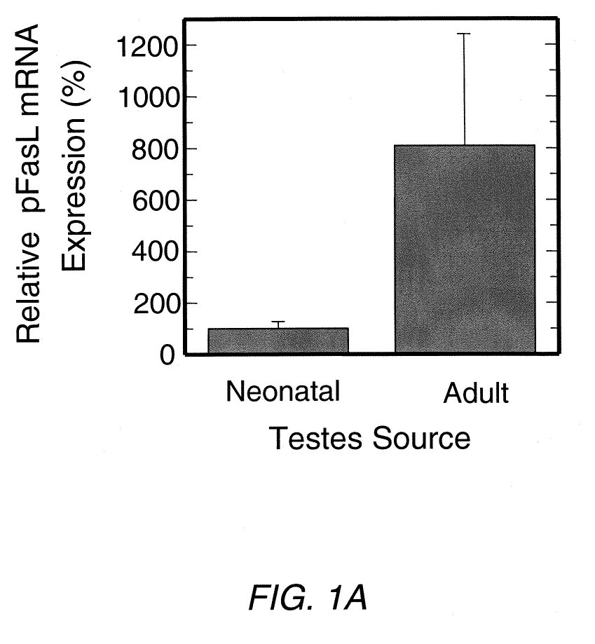

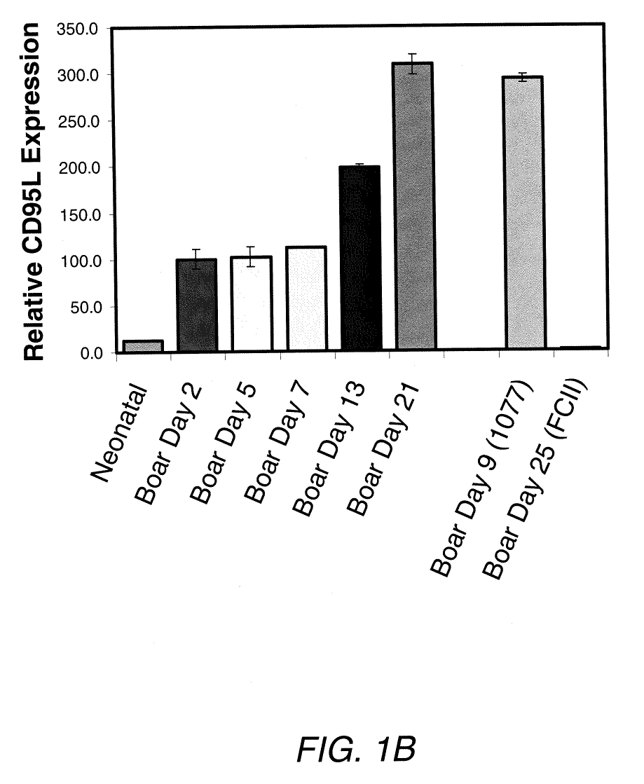

FIG. 1A shows relative expression of CD95L (pFasL) mRNA by RealTime PCR in fresh neonatal or adult porcine testicle. Adult testicles consistently showed approximately eight-fold higher expression levels than neonatal testicles. RealTime PCR was conducted as described.

FIG. 1B shows relative expression of porcine CD95L in cultured Sertoli cells. Boar cells were cultured in 10% bovine serum in high glucose DMEM media at 37° C., 5% CO₂, and 5% O₂ (for cell isolation see Example 2 below). Note that CD95L significantly increases over the culture period. Day 9 (1077) was prepared separately using the small cell isolation procedure (Histopaque gradient) and showed a significant increase in CD95L expression by the 7^{th} day. These levels were similar to those seen in the standard culture on Day 21. Cells from Day 25 (FCII) were grown continuously in 15% FetalClone II in DMEM high glucose and showed a nearly complete loss of CD95L expression.

### Example 2

### Co-Transplantation of Pig Islets with Adult Sertoli Cells into Rats Results in Lower Antibody Response than with Neonatal Sertoli Cells

Neonatal Porcine Islet Isolation

Pancreas Retrieval—Pancreata were obtained from a neonatal porcine heart beating donor. En bloc dissection using the no-touch technique was performed and pancreata were transported at 4° C. in sterile containers containing Hanks' Balanced Salt Solution transport media (HBSS transport media; 0.5% bovine serum albumin, 1% HEPES buffer solution, and 1% penicillin-streptomycin).

Islet Isolation—Pancreata were minced and mechanically digested with collagenase (2 ml/g pancreas; Liberase PI; Roche Applied Science, Indianapolis, Ind.) via continuous warm rigorous shaking (140 rpm for 15 min at 37° C.). Digested tissue was then strained through a 450 μm stainless steel mesh. Non-digested tissue was digested again (1 ml of Liberase PI per 1 g of remaining tissue) for 10 min. All fractions were combined and centrifuged at 1000 rpm for 1 min. Pellets were then washed 3 times in HBSS transport media. Islets were cultured in RPMI 1640 culture media supplemented with 0.5% BSA, 10 mmol/L nicotinamide, 1% penicillin-streptomycin.

See also Valdes-Gonzalez et al., Improved method for isolation of porcine neonatal pancreatic cell clusters. Xenotransplant. (2005) 12:240-244.

### Neonatal Porcine Sertoli Cell Isolation

Testicles were excised aseptically and placed in a sterile stainless steel pot containing sterile 0.9% saline slush. The vas deferens and epididymis were trimmed off from the testes, leaving the tunica albuginea intact. The tunica albuginea was then removed and the testes tissue weighed and minced into 1-2 mm fragments. The tissue was transferred to a 50 ml centrifuge tube with 30-40 ml of HBSS transport media. The contents of the tube were mixed by gently inverting 4 times, then allowed to sediment by gravity for 5 min. All but 5 ml of media above the pellet was removed and the tissue transferred to a sterile 100 ml Pyrex media bottle with 40 glass beads (2 mm). Digestion was carried out in HBSS (10 mL/g of testicle, without phenol-Red) containing 2.5 mg/ml collagenase and 0.15 mg/m DNase I solution in the shaking waterbath at 37° C. set to 200 rpm for 3-5 min. To determine the required length of the digestion, a 10 μl sample aliquot of the digest was mixed 1:1 with trypan blue after 3 min, and every 2 min thereafter. The reaction was stopped when the length of the tubules was 5150 μm. 30-40 ml of HBSS with FBS was added to inactivate the collagenase. The digest was sieved with a 400 μm. The samples were centrifuged at 400×g for 4 min at 4° C. The supernatant is removed and the cell pellet is resuspended in 50 mL of HBSS/FBS. The centrifugation and wash steps were repeated two more times, resulting in a total of 4 washes. Cells were resuspended in complete media (DMEM with 10% bovine serum and 1% penicillin/streptomycin), counted and viability checked with typan blue (typically >95%). 25−30×10⁶ isolated Sertoli cells were then cultured overnight in T75 culture flasks (Falcon) in 25-30 ml of complete culture media at 37° C. and 5% CO₂.

### Adult Porcine Sertoli Cell Isolation

Testicles were excised aseptically and the vas deferens and epididymis trimmed, leaving the tunica albuginea intact. Tissues were transported to the isolation facility on ice in HBSS transport media. Approximately 10 g of tissue was obtained from the testicle. The tissue was minced into 1-2 mm fragments and digestion was performed with 100 mL of filter sterilized (0.2 μm) 2.5 mg/ml collagenase (Type V, Sigma) and 0.15 mg/ml DNase I (Sigma) in HBSS (w/o phenol red, CellGrow). The tissue was transferred to 2 sterile 100 ml Pyrex media bottles each with 40 glass beads (2 mm) and incubated in a shaking water bath at 37° C. set to 200 rpm for 3-15 min. The reaction was stopped when the length of the tubules was ≦150 μm as determined by microscopic examination. Approximately 30-40 ml of HBSS with FBS was added to inactivate the collagenase and the digest was sieved with a 400 μm mesh. The cells were then transferred into 2×50 ml conical tubes and resuspended 4 times with a 10 ml pipette. The total volume was then brought to ˜45 ml per tube with the HBSS. The samples were centrifuged at 700 g for 15 min at 4° C. and the pellets were then washed 3 times with 50 mL of HBSS. Cells greater than 3 μm in diameter were counted and viability staining performed on all preparations using typan blue (viability typically >95%). 25×10⁶ cells (size >3 um) were seeded into 75 cm² culture flasks in 25-30 mL of complete media and incubated overnight at 37° C. and 5% CO₂.

### Rat Transplantation Studies

Animals—Female Lewis rats, weighing at least 200 g, were used as recipients (Charles River Canada). Animals were housed under conventional conditions at the Animal Care Facility of the University of Western Ontario and were cared for in accordance with the guidelines established by the Canadian Council on Animal Care.

Surgery—Four weeks prior to cell transplantation, recipients were transplanted with two polypropylene mesh chambers, 20 mm in length, containing a Teflon stent. Under general anesthesia, chambers were placed subcutaneously on the abdominal side of the animals and the skin sutured. On the day of cell transplantation, rats were anesthetized and a small incision was made to allow for removal of the Teflon stent from transplanted chambers. Cells were transplanted into the neovascularized collagen pouch, located within the chamber, the chamber was sealed using a Teflon screw cap and the incision sutured.

Four different treatment groups were established: 1) 2,000 neonatal islets plus 200,000 neonatal Sertoli cells into each chamber; 2) 2,000 neonatal islets plus 11×10⁶ neonatal Sertoli cells into each chamber; 3) 2,000 neonatal islets plus 200,000 adult Sertoli cells into each chamber; 4) 2,000 neonatal islets plus 11×10⁶ adult Sertoli cells into each chamber.

Binding Assay—Blood samples were collected weekly from the saphenous vein of the rat for 5 weeks post-transplantation. Blood was spun, and serum stored at −80° C. until the time of assay. On the day of assay, serum was heat inactivated for 30 minutes at 56° C. 2×10⁵ PK15 cells (ATCC) in 20 μl of serum free DMEM (Hyclone) were incubated with 20 μl of doubling dilutions of heat inactivated rat serum for 30 minutes at 4° C. Cell suspensions were washed in wash solution (phosphate buffered saline (MP Biomedical) containing 1% bovine serum albumin (EMD Science) and 0.01% sodium azide (VWR)). Fifty μl of goat anti-rat IgG (Invitrogen), used at a dilution of 1/400, was incubated with cells for 30 minutes at 4° C. Cell suspensions were washed twice in wash solution. Fluorescence of each sample was measured using a Cytomics FC500 flow cytometer (Beckman Coulter).

### Results

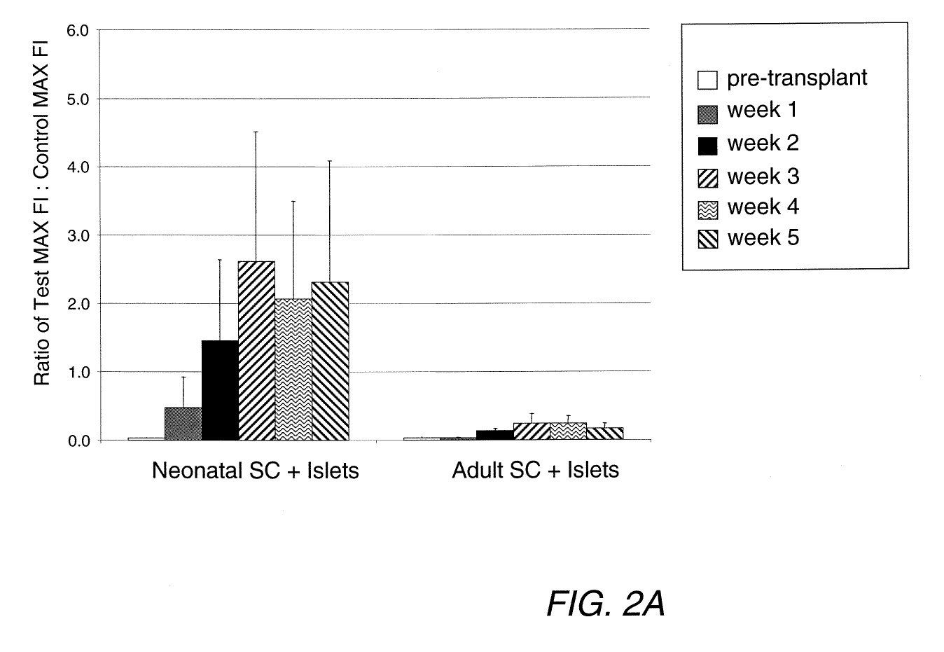

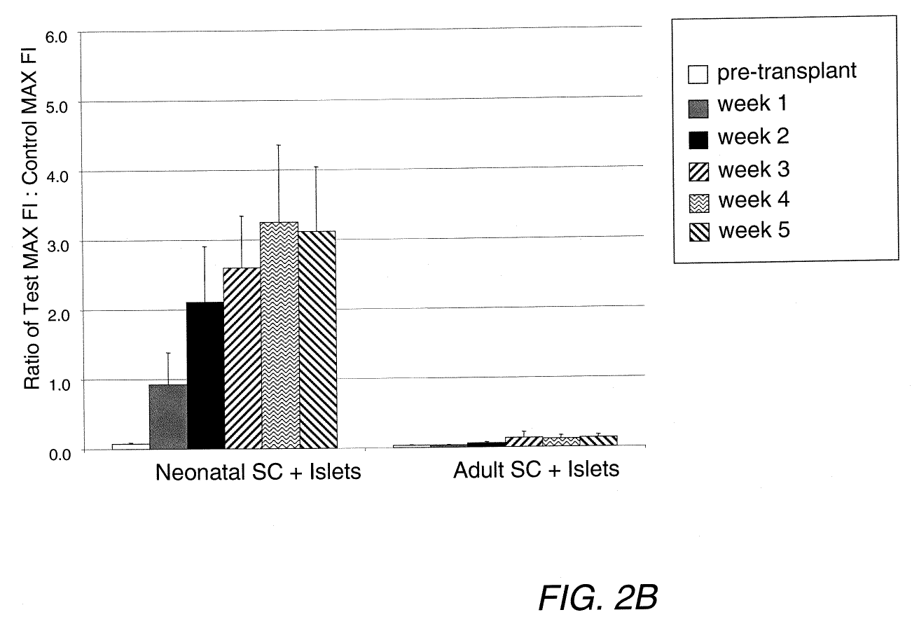

Neonatal or adult porcine Sertoli cells (SC) were mixed with neonatal porcine islets (200,000 SC/2,000 islets) and transplanted into non-immunosuppressed rats. Rat serum was collected at various time points as indicated and analyzed for the amounts of rat anti-pig IgG antibodies. Heat inactivated serum was incubated with PK15 cells (pig kidney cell line), followed by incubation with goat anti-rat antibody conjugated to a fluorophore. The amount of rat anti-pig IgG was quantified using flow cytometry. As shown in FIG. 2A, a significant drop in rat anti-pig IgG was observed when islets were co-transplanted with adult Sertoli cells compared to islet co-transplantation with neonatal Sertoli cells.

The experiment as described above was also performed with cells mixed at a ratio of 11×10⁶ SC/2,000 islets. A significant decrease in rat anti-pig IgG was observed when islets were co-transplanted with adult Sertoli cells compared to neonatal Sertoli cells (FIG. 2B). In addition, the anti-pig response to 11×10⁶ adult Sertoli cells was somewhat diminished when compared with the response for 200,000 adult Sertoli cells (FIG. 2A).

### Example 3

### Immunopathology Following Co-Transplantation of Pig Islets and Adult vs. Neonatal Sertoli Cells in a Collagen Pouch

### Methods

Surgery—Animals and protocols were as described in Example 2. Six different treatment groups were examined in one study:

1) 2,000 neonatal islets plus 200,000 neonatal Sertoli cells into each chamber;
2) 2,000 neonatal islets plus 11×10⁶ neonatal Sertoli cells into each chamber;
3) 2,000 neonatal islets plus 200,000 adult Sertoli cells into each chamber;
4) 2,000 neonatal islets plus 11×10⁶ adult Sertoli cells into each chamber;
5) 2,000 neonatal islets alone; and

6) 200,000 neonatal Sertoli cells alone.

In another study, three other treatment groups were examined:

1) 4,000 porcine islets alone;
2) 4,000 porcine islets plus 400,000 neonatal Sertoli cells; and
3) 4,000 porcine islets plus 400,000 adult Sertoli cells.

Tissue Collection—All rats were sacrificed in a CO₂ chamber 1 day to 6 weeks post cell transplant. Chambers were removed from sacrificed animals and were fixed in 10% buffered neutral formalin (VWR). After at least three days, the chambers were cut in cross section and embedded in paraffin.

Hematoxylin/Eosin Staining—Five micron sections were cut and placed onto poly-L-lysine glass slides (Fisher). The slides were then dewaxed and rehydrated. Sections were stained with hematoxylin (Surgipath) for 5 minutes. They were then dipped 5 times in 1% acid alcohol followed by 10 dips in 1% ammonia acid. Tissue was counter stained using eosin (Surgipath) for 2 minutes after which it was dehydrated, cleared and mounted.

Immunostaining—Five micron sections were cut and placed onto poly-L-lysine glass slides (Fisher). The slides were then dewaxed and rehydrated. Antigen retrieval was performed through incubation of the slides with EDTA, pH 8.0 at high pressure for 3 minutes. Incubating the slides in a 3% solution of H₂O₂ for 10 minutes blocked nonspecific binding. The sections were incubated with monoclonal mouse anti-insulin (Novastra) at a 1:50 dilution for 1 hour. Sections were incubated with the secondary antibody anti-mouse envision system (Dako) for 30 minutes. DAB (Dako) was used as a substrate for colour development. The sections were then counter stained with hematoxylin (Dako) for 5 minutes, dehydrated, cleared and mounted.

### Results

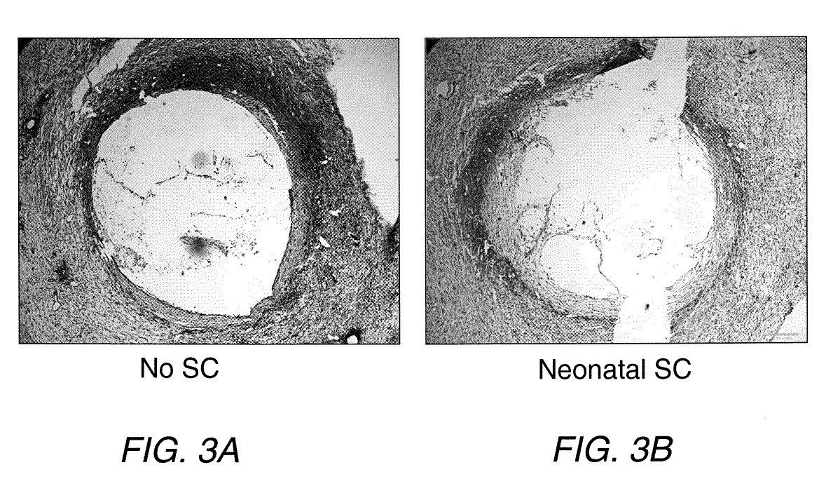

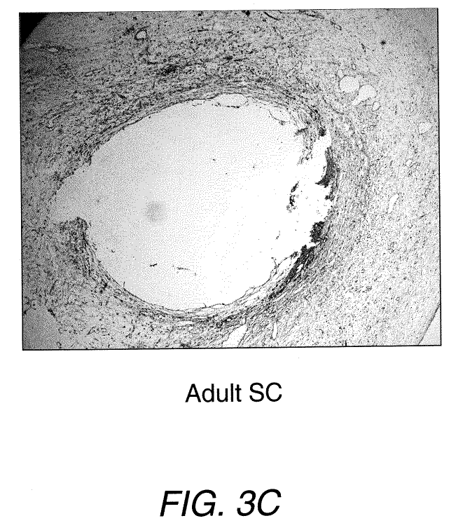

FIGS. 3A, 3B and 3C. Non-immunosuppressed rats were transplanted with either 2,000 neonatal porcine islets (FIG. 3A) or 2,000 neonatal porcine islets and 200,000 neonatal porcine Sertoli cells (FIG. 3B) or 2,000 neonatal porcine islets and 200,000 adult porcine Sertoli cells (FIG. 3C) into neovascularized chambers. On day 7 post-transplantation, animals were sacrificed, chambers were removed and tissue sections examined by H&E staining. A cellular infiltrate was observed in transplants of islets alone and islets co-transplanted with neonatal Sertoli cells (FIG. 3A and FIG. 3B). However, a diminished response was observed when islets were co-transplanted with neonatal Sertoli cells (FIG. 3B) and completely abolished when islets were co-transplanted with adult Sertoli cells (FIG. 3C).

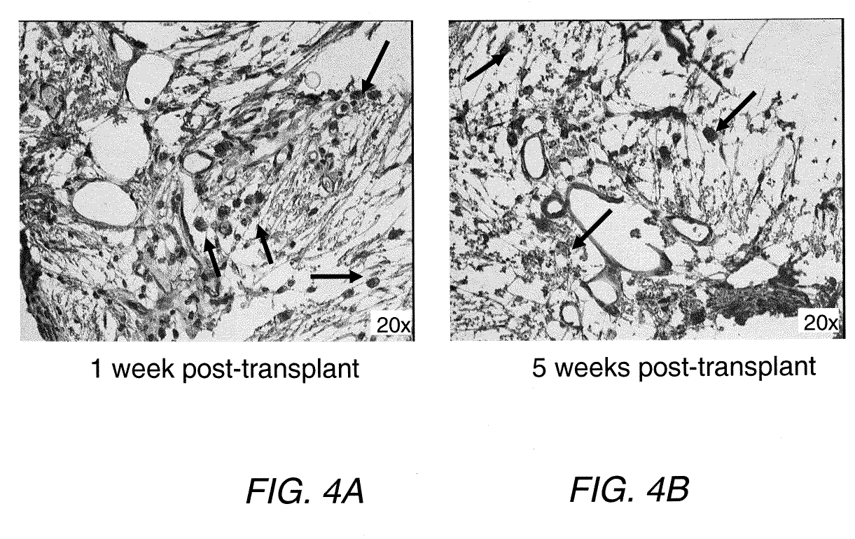

FIGS. 4A and 4B. Non-immunosuppressed rats transplanted with 2,000 islets and 200,000 neonatal Sertoli cells in neovasularized chambers were examined for the presence of insulin positive cells at 1 week and 5 weeks post-transplantation as described above. Positive cells were observed at both 1 week (FIG. 4A) and remained at 5 weeks (FIG. 4B) post-transplantation.

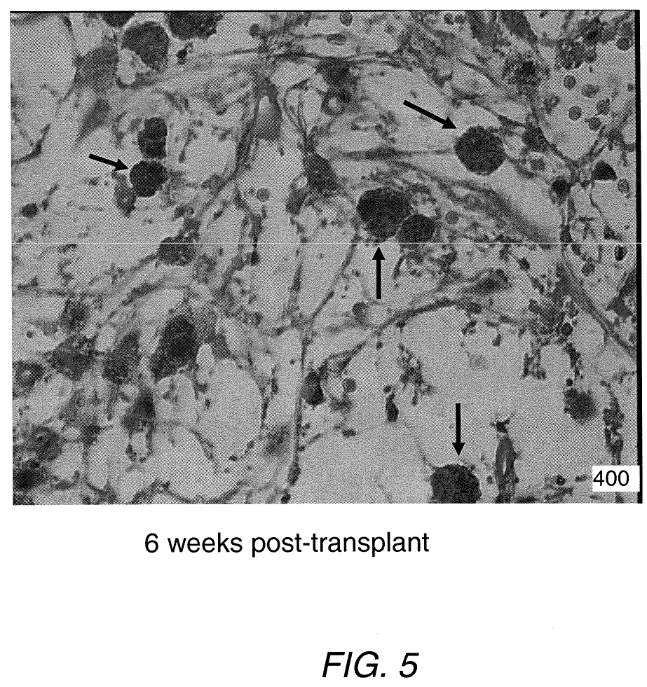

FIG. 5. Non-immunosuppressed rats transplanted with 200,000 adult Sertoli cells and 2,000 neonatal islets in neovascularized chambers were examined for the presence of insulin positive cells as described above. At 6 weeks post-transplantation, a large number of positive cells were observed.

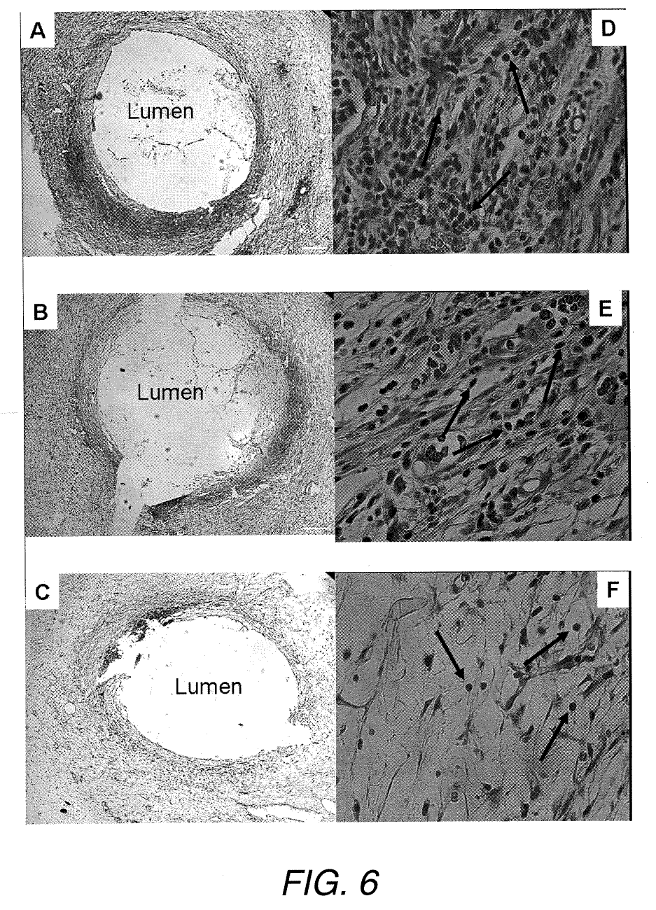

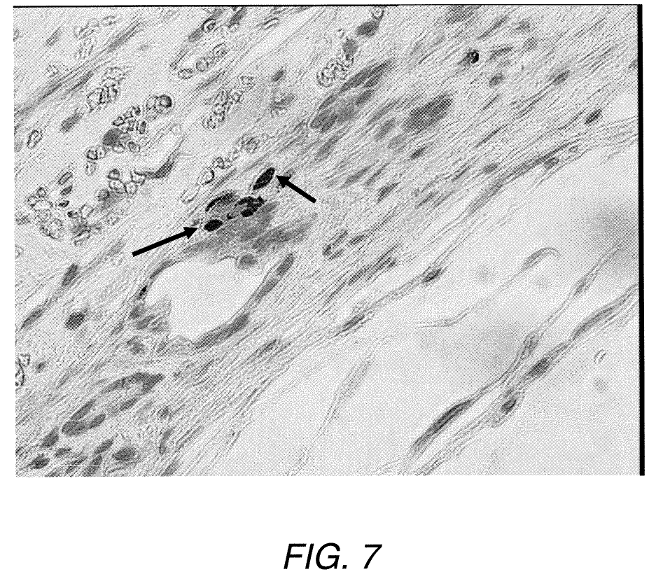

FIGS. 6A, 6B, 6C. 6D, 6E, 6F, and 7: Non-immunosuppressed rats were transplanted with either 4,000 porcine islets (FIGS. 6A and 6D), 4,000 islets plus 400,000 neonatal Sertoli cells (FIGS. 6B and 6E), or 4,000 islets plus 400,000 adult Sertoli cells (FIGS. 6C, 6F, and 7). Chambers were removed from rats 7 days post-transplantation and stained with hematoxylin and eosin (FIGS. 6A to 6F). As shown in FIG. 6A, after 7 days, a large infiltrate of inflammatory cells was present when 4,000 islets alone were transplanted into rats. At a higher magnification (FIG. 6D), the infiltrating cells appear as densely packed mononuclear cells. Co-transplantation of neonatal Sertoli cells with islet cells at a ratio of 100 Sertoli cells for every islet cell resulted in a decrease in the number of inflammatory cells infiltrating the graft (FIG. 6B). These mononuclear cells were somewhat less densely packed (FIG. 6E) compared to those that infiltrated islets transplanted alone. When adult Sertoli cells were used in combination with islets (at a ratio of 100 Sertoli cells for each islet) the number of inflammatory cells was minimal (FIG. 6C). While there were a few mononuclear cells detectable at higher magnification (FIG. 6F), the cells were present at a much lower density when compared to mononuclear cells that infiltrated islet-only transplants, or the islet plus neonatal Sertoli cell co-transplants.

Chambers removed from rats 7 days post-transplantation were also stained for insulin by immunohistochemistry as follows. Five micron sections were cut and placed onto poly-L-lysine glass slides (VWR). The slides were then dewaxed and rehydrated. Antigen retrieval was performed through incubation of the slides with EDTA, pH 8.0 at high pressure for 3 minutes. The slides were next incubated in a 3% solution of H₂O₂ for 10 minutes to block any endogenous peroxidase activity. The sections were incubated with monoclonal mouse anti-insulin (Novocastra, Norwell, Mass.) at a 1:75 dilution for 1 hour. Sections were incubated with the secondary antibody anti-mouse ABC (Avidin: Biotinylated enzyme Complex) system (Vector Laboratories, Burlington, ON) for 60 minutes. An AEC substrate kit (3-amino-9-ethylcarbazole, Vector Laboratories) that was compatible with the peroxidase enzyme present in the ABC system was used to give a red reaction product. The sections were then counter stained with hematoxylin (Dako, Mississauga, ON) for 5 minutes, dehydrated, cleared and mounted. Only transplants of 4,000 islet cells and 400,000 adult Sertoli cells stained positive for porcine insulin in this study, as indicated by the dark-stained cells in FIG. 7.

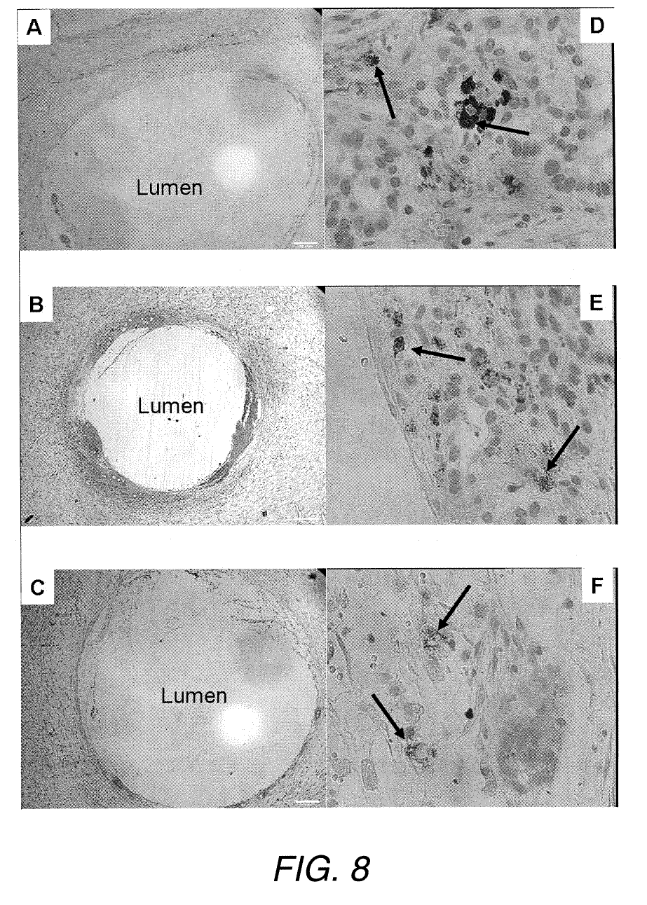

FIGS. 8A, 8B, 8C, 8D, 8E, and 8F: Non-immunosuppressed rats were transplanted with either 4,000 porcine islets (FIGS. 8A and 8D), 4,000 islets plus 400,000 neonatal Sertoli cells (FIGS. 8B and 8E), or 4,000 islets plus 400,000 adult Sertoli cells (FIGS. 8C and 8F). Chambers were removed from rats 4 days post-transplantation and either stained with hematoxylin and eosin (FIGS. 8A, 8B, and 8C) or stained for insulin by immunohistochemistry (FIGS. 8D, 8E, and 8F) as described for FIG. 7.

When transplanted chambers were removed from rats 4 days after transplantation, there was very little evidence of an immune response as demonstrated by the lack of infiltrating inflammatory cells (FIGS. 8A to 8F) in any of the treatment groups. Insulin positive cells were present in all three treatment groups, as demonstrated by the dark stained cells in FIGS. 8D, 8E, and 8F.

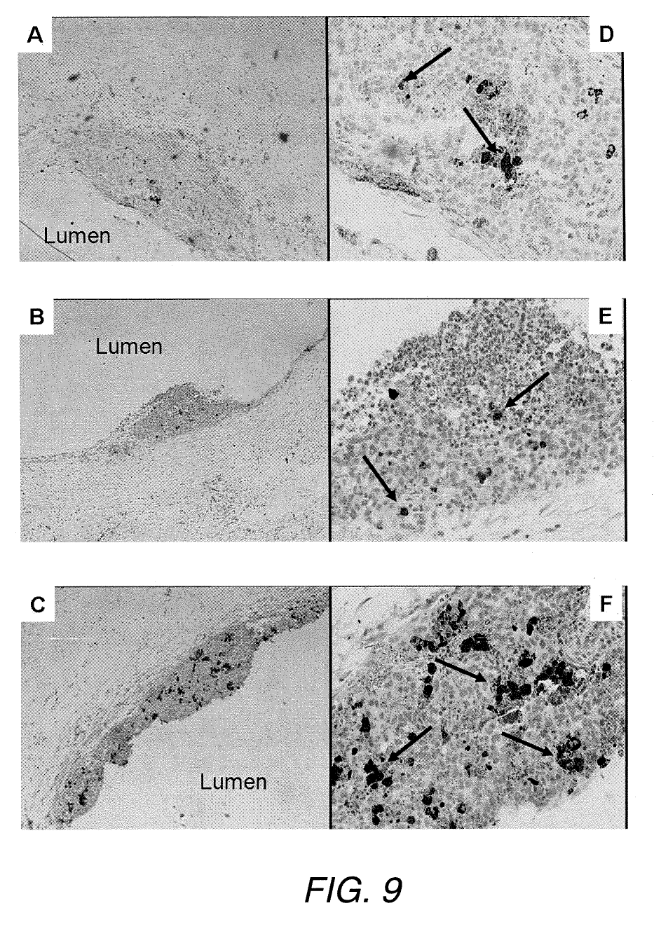

FIGS. 9A, 9B, 9C, 9D, 9E, and 9F: Non-immunosuppressed rats were transplanted with either 4,000 porcine islets (FIGS. 9A and 9D), 4,000 islets plus 400,000 neonatal Sertoli cells (FIGS. 9B and 9E), or 4,000 islets plus 400,000 adult Sertoli cells (FIGS. 9C and 9F). Chambers were removed from rats 1 day after transplantation and stained for insulin by immunohistochemistry as described above for FIG. 7. Insulin positive cells were detected in all three treatment groups 1 day after transplantation.

### Example 4

### Production of Insulin by Neonatal Porcine Islets Co-Transplanted with Adult Sertoli Cells

Human insulin ELISA is a method that provides quantitative determination of porcine insulin in vivo. Because human insulin and porcine insulin differ by only one amino acid, this particular assay has proven useful for porcine insulin detection with rat insulin detection <1%. For additional information, see, e.g., Jay et al., Transplant. Proc. (2004) 36(4):1130-32; Lakey et al., Transplantation (2002) 73(7):1106-10.

Non-immunosuppressed rats were implanted with two chamber devices, as described in Example 2. Four weeks later the animals were transplanted with 2000 neonatal porcine islet cells and 11 million adult porcine Sertoli cells in each chamber. Serum samples were obtained from these animals 1 and 2 weeks post-transplantation (non-fasted). Using the Human Insulin ELISA kit (Mercodia/ALPCO) per manufacturer's instructions, porcine insulin was detected 1 and 2 weeks post-transplantation with greatest levels demonstrated at 2 weeks post-transplantation.

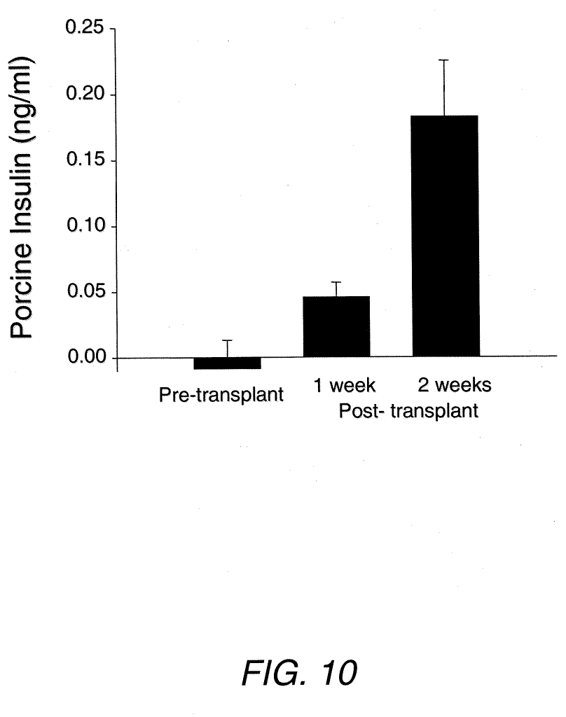

Porcine insulin levels in non-fasted, non-immunosuppressed rats transplanted with porcine islets and adult Sertoli cells were measured by ELISA. As shown in FIG. 10, pre-transplantation levels of porcine insulin were negative. The presence of physiological levels of porcine insulin was evident at 2 weeks post-transplantation, suggesting the survival of functioning porcine beta cells in the polypropylene mesh chambers.

### Example 5

### CD95L (FasL) Expression Profile in Primary Neonatal and Adult Sertoli Cells Isolated from Tissue

Two-day neonatal pig testicles were decapsulated, minced and initially digested with collagenase V in HBSS for 10 minutes with shaking at 37° C. Following the digestion, the tissue was washed several times with HBSS and finally was suspended in cell dissociation buffer containing 0.33 μg/ml trypsin and 0.02 μg/ml DNase I. The tissue was incubated for 10 minutes at 37° C. in a shaking water bath. The digested tissue was passed thru a 420 micron filter to obtain the neonatal Sertoli cells which were subsequently cultured in Ham's F10 supplemented with 0.5% BSA, 10% Fetal Bovine Serum, 100 ug/ml Penicillin and Streptomycin, 50 ug/ml Gentamycin Sulfate, 10 mM Nicotinamide, 2 mM L-Glutamine, 50 uM 3-Isobutyl-1-methyl-xanthine (IBMX) in a humidified 5% CO₂ atmosphere incubator at a temperature of 37° C. The neonatal Sertoli cells were cultured for two days and then lysed in protein lysis buffer (0.5% Triton X-100, 150 mM NaCl, 50 mM Tris-Cl, pH 7.5, 1 mM Phenylmethylsulfonyl Fluoride (PMSF), 5 ug/mL Aprotinin, 1 ug/mL Pepstatin A and 1 mM Sodium ortho-vanadate), cleared and loaded onto a 12% SDS-PAGE gel. The proteins were then transferred to PVDF membranes and probed with either 1:500 FasL antibody (Cell Signaling Technology) or 1:100 β-tubulin antibody (generous gift from Dr. Lina Dagnino). The blots were washed and further incubated with the appropriate secondary HRP-conjugated antibody. The Western blots were exposed using enhanced chemiluminescence (Pierce).

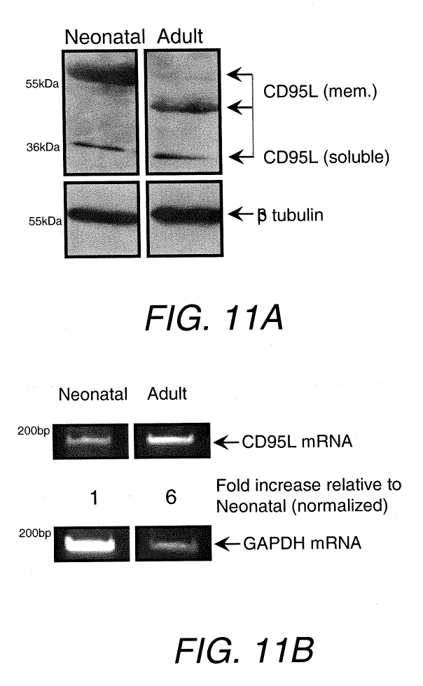

Adult porcine testicle tissue was obtained and homogenized with short pulses in a solution of 10M urea, 150 mM NaCl, 50 mM Tris-Cl, pH 7.5, and protease and phosphatase inhibitors as described above. The solubilized adult tissue extract was cleared and was handled in a similar fashion as described above for the neonatal Sertoli cells to obtain a CD95L and β-tubulin Western blot profile (FIG. 11A). As shown in FIG. 11A, the membrane-bound form of CD95L in adult tissue appears to be different from that found in neonatal tissue, based on a mobility shift of the protein in the Western Blot. This change in observed mobility could be due to a difference in phosphorylation and/or glycosylation on CD95L between the neonatal and adult testicular tissues.

An aliquot of the neonatal Sertoli cells obtained as described above for the Western blot analysis were lysed and total RNA was isolated using the Mini RNA kit from Qiagen. One μg of total RNA was reversed transcribed into cDNA for amplification. An aliquot of the reverse-transcribed mRNA was amplified with primers for either CD95L (primers qF1: 5′ACT GAA CTC AGA GAG TCT GCC AGC C (SEQ ID NO:1) and qR1: 5′GGA TGG ATC TTG AGT TAG GCT TGC C (SEQ ID NO:2)) or pig GAPDH primers (Forward primer GTCCTCTGACTTTAACAGTGACACTCACTCTTCT (SEQ ID NO:9); Reverse primer=CCACCCTGTTGCTGTAGCCAAATTCATTGTCGTACG (SEQ ID NO:10) using the Qiagen Fast cycling PCR kit using the following conditions: 35 cycles of 95° C. for 30 seconds, 58° C. annealing for 5 seconds, 68° C. extension for 15 seconds. The PCR product was resolved on a 12% acrylamide gel and stained with ethidium bromide for 10 minutes. A digitized image of the gel was captured using an Image Capture station.

A piece of adult pig testicular tissue obtained as described above for the Western blot analysis was homogenized to obtain RNA using the Qiagen Mini total RNA kit by following the manufacturer's instructions. The adult tissue RNA was handled in a similar way as the neonatal tissue RNA to obtain PCR amplified CD95L and GAPDH cDNA. The digitized image of the amplified CD95L and GAPDH cDNA, resolved by gel electrophoresis, was saved as a tif image and then quantified using ImageQuant version 5.2 imaging software (Molecular Dynamics). The levels of GAPDH amplified product was first normalized between neonatal and adult tissues and a relative fold increase over neonatal tissue was calculated (FIG. 11B). Normalized CD95L mRNA levels were at least 6-fold higher in adult testicle tissue compared to neonatal tissue.

All publications and patent documents cited herein are incorporated by reference in their entirety. To the extent the material incorporated by reference contradicts or is inconsistent with the present specification, the present specification will supersede any such material.

1. A pharmaceutical composition comprising adult, non-rodent, non-human Sertoli cells and a pharmaceutically acceptable carrier.

2. The composition of claim 1, wherein the adult Sertoli cells express more FasL at the RNA and/or at the protein level(s) than neonatal Sertoli cells of the same species.

3. (canceled)

4. The composition of claim 1, wherein the adult Sertoli cells are porcine cells.

5. (canceled)

6. The composition of claim 1, wherein the adult Sertoli cells are primate cells.

7. (canceled)

8. (canceled)

9. The composition of claim 1, wherein the adult Sertoli cells are primary cells.

10. The composition of claim 1, wherein the adult Sertoli cells provide greater immunoprivilege than neonatal Sertoli cells of the same species.

11. The composition of claim 1, wherein the adult Sertoli cells are modified to express a biological factor.

12. The composition of claim 11, wherein the biological factor is insulin.

13. The composition of claim 1, wherein the composition further comprises non-Sertoli cells.

14. The composition of claim 1, wherein the non-Sertoli cells are insulin-secreting cells.

15. The composition of claim 14, wherein the insulin-secreting cells are beta cells.

16. The composition of claim 14, wherein the insulin-secreting cells are modified hepatocytes.

17. An implantation device comprising the pharmaceutical composition of claim 1.

18. The device of claim 17, wherein the device is adapted to induce formation of a fibrotic capsule when implanted into a mammal.

19. A method of making the composition of claim 1, comprising isolating Sertoli cells from an adult, non-rodent, non-human mammal.

20. (canceled)

21. A method of using the composition of claim 1, comprising administering an effective amount of the composition to a subject.

22. The method of claim 21, wherein the Sertoli cells are administered in a device or to a site with a pre-implanted device.

23. The method of claim 21, wherein the Sertoli cells are allogeneic to the subject.

24. The method of claim 21, wherein the Sertoli cells are xenogeneic to the subject.

25. The method of claim 21, wherein the subject is human.

26. The methods method of claim 21, wherein the subject is a non-human mammal.

27. The method of claim 21, wherein the subject has diabetes.

28. A method of treating diabetes, comprising co-administering adult non-human, non-rodent Sertoli cells and islets cells to a mammal in need thereof and under conditions that allow islet cells to survive and produce insulin subsequent to the administration.

29. A method of selecting adult Sertoli cells with increased immunoprotective properties, the method comprising determining the amount of FasL expressed by the Sertoli cells, and selecting cells expressing higher amounts of FasL.


SEQUENCE LISTING (10 sequences)

SEQ ID NO: 1 - DNA, 25 bp, Artificial
  Primer Sequence
actgaactcagagagtctgccagcc

SEQ ID NO: 2 - DNA, 25 bp, Artificial
  Primer sequence
ggatggatcttgagttaggcttgcc

SEQ ID NO: 3 - DNA, 25 bp, Artificial
  Primer sequence
tgatgttcttcatggttctggtggc

SEQ ID NO: 4 - DNA, 28 bp, Artificial
  Primer sequence
gcttctccaaagatgattctgtatgcct

SEQ ID NO: 5 - DNA, 27 bp, Artificial
  Primer sequence
tcttccacctacagaaggagctgactg

SEQ ID NO: 6 - DNA, 25 bp, Artificial
  Primer sequence
ccattccagagggatggatcttgag

SEQ ID NO: 7 - DNA, 21 bp, Artificial
  Primer sequence
ttgccgacaggatgcagaagg

SEQ ID NO: 8 - DNA, 21 bp, Artificial
  Primer sequence
gacagcgaggccaggatggag

SEQ ID NO: 9 - DNA, 34 bp, Artificial
  Primer sequence
gtcctctgactttaacagtgacactcactcttct

SEQ ID NO: 10 - DNA, 36 bp, Artificial
  Primer sequence
ccaccctgttgctgtagccaaattcattgtcgtacg